(19) United States
(12) Patent Application Publication
(10) Pub. No.: US 2022/0313727 A1
(43) Pub. Date: Oct. 6, 2022

(54) INHALABLE GASEOUS MIXTURE FOR TREATING CHRONIC PAIN IN PATENTS RECEIVING DRUGS FROM MULTIPLE THERAPEUTIC CLASSES

(71) Applicant: L'Air Liquide, Société Anonyme pour l'Etude et l'Exploitation des Procédés Georges Claude, Paris (FR)

(72) Inventors: Laurent LECOURT, Cachan (FR); Baptiste BESSIERE, Issy les Moulineaux (FR); Patrick HOUETO, Ermont (FR); Céclle DELVAL, Les Loges-en-Josas (FR); Juan Ferando RAMIREZ, Antony (FR)

(21) Appl. No.: 17/607,965

(22) PCT Filed: Oct. 10, 2019
(86) PCT No.: PCT/FR2019/052400

§ 371 Date: Nov. 1, 2021

(30) Foreign Application Priority Data
May 2, 2019 (FR) 1904626

(51) Int. Cl. A61K 33/00; A61P 25/02; A61K 9/00

(52) U.S. Cl. CPC A61K 33/00; A61P 25/02; A61K 9/007

(57) ABSTRACT

The invention relates to a gaseous mixture containing 50% of nitrous oxide (N₂O) and at least 20% of oxygen (O₂) (mol %) for use as an inhalable medicament for treating chronic pain in a human patient treated with a plurality of drug substances of at least two different therapeutic classes.

### CROSS REFERENCE TO RELATED APPLICATIONS

This application is a 371 of International Application No. PCT/FR2019/052400, filed Oct. 10, 2019, which claims priority to French Patent Application No. 1904626, filed May 2, 2019, the entire contents of which are incorporated herein by reference.

### BACKGROUND

The invention relates to a gas mixture containing 50% of nitrous oxide (mol %) and oxygen, preferably 50% of oxygen (mol %), used as an inhalable drug for therapeutically treating patients continuing to experience chronic pain, in particular chronic peripheral neuropathic pain, despite taking at least two other different pain treatments, i.e. drug substances or compounds belonging to at least two classes different therapeutic classes, in particular patients who are refractory to said drug substances or compounds belonging to at least two different therapeutic classes.

Chronic pain can have a significant impact on the quality of life of many patients, in particular their functional activity, their sleep, their levels of anxiety and depression, etc. As a result, patients suffering from chronic pain, in particular from peripheral neuropathies, must be treated therapeutically, that is to say, must follow basic drug treatments aimed at reducing or eliminating their pain.

To this end, it is recommended that they first be administered first-line drug treatments, namely tricyclic antidepressants, serotonin-norepinephrine reuptake inhibitor antidepressants (e.g. duloxetine) or even antiepileptics which act on sodium or calcium channels (e.g. gabapentin, pregabalin, etc.).

However, it has been found that these drugs are not efficacious in some patients since the pain persists despite taking these drugs. Moreover, other patients do not tolerate these treatments because these drugs lead in these patients to negative side effects, such as nausea, vomiting, drowsiness, dizziness, respiratory distress, etc. In point of fact, these side effects are difficult to bear, i.e. poor tolerance.

It is then necessary to resort to other drugs, such as for example opioids, or compounds or substances which are targeted more locally, such as qutenza.

However, the more patients are treated, the less they generally respond to new treatments. In addition, these patients are also the most vulnerable and therefore often have difficulty tolerating combinations of different treatments/drugs, in particular combinations of drug substances or drug compounds belonging to at least two different therapeutic classes, in particular combinations between opioids, antidepressants, antiepileptics and/or other local anesthetics.

In other words, it is difficult to provide relief to certain particular categories of patients suffering from chronic pain, in particular from peripheral neuropathies, and who are treated with combinations of different treatments, that is to say who are receiving drug substances or drug compounds from at least two different therapeutic classes because these patients have poor tolerance to these combinations and/or are less responsive to these combinations, in particular combinations between opioids, antidepressants, antiepileptics and/or other local anesthetics.

Moreover, FR-A-2981276 is known which describes the use of xenon or of N₂O for treating particular neuropathic pain, namely pain induced by platinum salts (i.e., cisplatin, oxaliplatin, carboplatin) or another anticancer substance in a particular patient population, namely patients suffering from cancer. Contents ranging up to 70% by volume of xenon or of N₂O are given, but it is specified that the preferential contents are less than 30% by volume.

In addition, FR-A-2944969 teaches the use of N₂O for treating neuropathic pain in a patient, in a content of between 5 and 45% by volume, preferably less than 35% by volume.

None of these documents relates to the particular patient population mentioned above, namely patients receiving and/or having already received at least two treatments (i.e. drug substances or compounds) from different therapeutic classes, i.e. patients suffering from chronic pain, in particular peripheral neuropathic pain, and already treated by repeated administration of at least two drug substances from different therapeutic classes, whether these treatments have been carried out in the past and/or are still ongoing, in particular combinations of opioids, antidepressants, antiepileptics and/or other local anesthetics.

One problem is therefore that of being able to provide an improved drug making it possible to effectively provide relief to patients suffering from chronic pain, in particular chronic peripheral neuropathic pain, and who are otherwise treated by administration from at least two drug substances from different therapeutic classes, that is to say a drug combination of several therapeutic compounds, whether or not these drug substances are still being administered to the patients, preferably with the drug acting over several weeks, this being outside any anesthesia procedure.

In other words, there is a persistent medical need in the management of chronic pain, in particular chronic peripheral neuropathic pain, in particular by treatments that are more efficacious and better tolerated by patients than current strategies, in particular in the population of patients already treated with at least two drug substances (i.e. compound or product) from different therapeutic classes, in particular patients who are refractory to treatments with said at least two drug substances from different therapeutic classes, this being in particular with a view to improving the benefit/risk ratio of such treatments.

In other words, it is desirable to be able to improve the efficacy and tolerance of patients suffering from chronic pain, in particular from chronic peripheral neuropathic pain, and treated with at least two drug substances (i.e. compound or product) from different therapeutic classes used for the treatment of chronic pain, which patients are refractory to said at least two drug substances from different therapeutic classes.

### SUMMARY

A solution according to the invention relates to a gas mixture containing 50% of nitrous oxide (N₂O) and at least 20% of oxygen (O₂) (mol %) for use as an inhalable drug for treating chronic pain in a human patient treated with several drugs from at least two different therapeutic classes, preferably a patient refractory to said drugs from at least two classes different therapeutic classes.

In other words, the gas mixture containing 50% of nitrous oxide (N₂O) and at least 20% of oxygen (O₂) (mol %) is used in the context of the invention as an inhalable drug for treating chronic pain in a human patient who has already been treated with several types of drug compounds belonging to different therapeutic classes which also have an action against chronic pain but which have shown little or no efficacy and/or which have led to intolerance in the patient in question, that is to say a patient refractory to these treatments.

In addition, surprisingly, when a gas mixture containing 50% N₂O and at least 20% of oxygen O₂, in particular EMONO, according to the invention was administered to patients previously treated with several types of drug compounds belonging to different therapeutic classes, the results in terms of efficacy and tolerance are better than those obtained in patients treated with only one of these drug compounds.

In the context of the present invention:
a refractory patient is a person in whom a treatment based on drug compounds belonging to different therapeutic classes, which has been administered to said patient beforehand for a long period of time, typically several days, weeks or months, in an attempt to relieve his or her chronic pain, failed to produce a sufficient effect to reduce said chronic pain, therefore in whom the drug compounds belonging to different therapeutic classes have little or no efficacy and/or are poorly tolerated, in particular because of possible side effects linked to a dosage regime incompatible with the patient in question;
the drug substances are administered as a background treatment for pain, in particular for long periods of time, typically for several days, weeks or months, preferably several months, or even years, with one or more daily administrations of these drug compounds; however, this background treatment with drug substances belonging to different therapeutic classes does not form part of the present invention;
the proportions of compounds (N₂O, O₂ . . . ) in the gas mixture are expressed in molar percentages (mol %);
the terms “drug substance”, “drug product” and “drug compound” are considered to be fully equivalent and are used interchangeably.

According to the embodiment considered, the gas mixture used as inhalable drug according to the invention may comprise one or more of the following characteristics:
it contains at least 30% of oxygen, preferably at least 40% of oxygen;
it contains a maximum of approximately 50% of oxygen;
it contains oxygen, N₂O and nitrogen (N₂);
it consists of 50% of nitrous oxide (N₂O) and 50% of oxygen (O₂). This equimolar (50%/50%) mixture is also referred to as “EMONO” for Equimolar Mixture of Oxygen and Nitrous Oxide;
of course, even though this is neither desirable nor desired, the N₂O/O₂ gas mixture according to the invention, such as EMONO, may optionally contain unavoidable impurities, for example impurities of the CO, CO₂, H₂O and NOₓ type. However, care is taken to ensure that the impurity level is as low as possible, in particular less than approximately 1%, typically less than 500 ppm, for example less than 300 ppm for impurities of CO and CO₂ type, less than 5 ppm for impurities of H₂O type and less than 2 ppm for impurities of NOₓ type;
nitrous oxide is the active ingredient;
the drug substances from at least two different therapeutic classes are selected from opioids, antiepileptics (AEs), antidepressants (ADs), and local anesthetics;
the patient is a human being, namely a man or a woman, whether a child, a teenager, an adult, including the elderly;
the patient does not respond or responds poorly to drug substances from at least two different therapeutic classes which have been administered to him or her;
the patient is refractory to drug substances from at least two different therapeutic classes which have been administered to him or her;
the human patient suffers from chronic pain;
the patient has developed an intolerance to at least one of the drug substances from at least two different therapeutic classes which have been administered to him or her;
the patient receives the gas mixture of nitrous oxide and oxygen concomitantly with, i.e. at the same time as, the drug substances from at least two different therapeutic classes;
the patient receives the gas mixture of nitrous oxide and oxygen subsequently to, i.e. after, the drug substances from at least two different therapeutic classes. However, this does not mean that the drug substances must be taken in combination throughout the duration of the treatment with the gas mixture of nitrous oxide and oxygen;
the drug substances from at least two different therapeutic classes are selected from opioids, antiepileptics (AEs) and antidepressants (ADs);
the drug substances from at least two different therapeutic classes comprise an antiepileptic compound selected from gabapentin and pregabalin;
the drug substances from at least two different therapeutic classes comprise an antidepressant compound:
   of the tricyclic type, preferably nortriptyline, desipramine or amitriptyline, or
   of the serotonin-norepinephrine reuptake inhibitor type, preferably duloxetine;
the drug substances from at least two different therapeutic classes comprise an opioid compound selected from tramadol, codeine, oxycodone, tapentadole, fentanyl, remifentanyl and morphine, i.e. morphine itself or any other natural or chemical morphine derivative;
the drug substances from at least two different therapeutic classes comprise a local anesthetic compound selected from lidocaine and capsaicin;
the chronic pain is neuropathic pain:
   the chronic pain has at least one peripheral component, for example mixed pain;
the chronic pain is peripheral neuropathic pain;
the chronic pain is peripheral neuropathic pain associated with and/or resulting from one (or more) lesions or irritations of one or more peripheral nerves;
the lesion or irritation of the peripheral nerve(s) is of traumatic, toxic, metabolic, ischemic, immunoallergic or infectious origin;
   the chronic peripheral neuropathic pain results from or is related to a lesion or irritation of one or more peripheral nerves;
the chronic peripheral neuropathic pain is selected in particular from postherpetic pain, diabetic neuropathy pain, polyneuropathy, and post-traumatic or post-operative pain;
the pain is spontaneous and/or provoked pain;
the spontaneous pain is continuous and/or superficial (e.g. burning sensation) or deep (e.g. feeling of tightening by a vice or compression), with or without occurrence of breakthrough pain (i.e. sensation of electric shock) and paresthesia/dysesthesia;
the provoked pain is characterized by hypersensitivity to heat, to cold or to touch;
the gas mixture is not administered during anesthesia, that is to say it is intended for conscious, i.e. non-anesthetized, patients. In other words, the gas mixture according to the invention is not intended for use in the context of an anesthesia procedure in patients having to undergo such an anesthesia procedure;
the gas mixture is packaged in a gas container, in particular a gas cylinder.

The invention also relates to a use of an N₂O/O₂ gas mixture containing 50% of nitrous oxide (N₂O) and at least 20% of oxygen (O₂) (mol %) according to the invention, preferably an equimolar 50%/50% N₂O/O₂ mixture, for producing an inhalable drug making it possible to effectively provide relief to patients suffering from chronic pain, in particular chronic peripheral neuropathic pain (CPNP), said patients being or having been treated with several drug substances from at least two different therapeutic classes, in particular antiepileptic, antidepressant, opiate (i.e. opioid) and/or local anesthetic drug substances, in particular patients refractory to said drug substances from at least two different therapeutic classes.

Furthermore, the invention also relates to a treatment method which makes it possible to provide relief to a patient suffering from one or more types of chronic pain, in particular chronic peripheral neuropathic pain (CPNP), and needing an additional treatment to at least partially relieve his or her chronic pain, wherein:

a) a patient receiving and/or having received, in an attempt to treat this chronic pain, several drug substances from at least two different therapeutic classes is selected, that is to say a patient in a more advanced stage of the disease, preferably patients refractory to said drug substances from at least two different therapeutic classes, and

b) an N₂O/O₂ gas mixture containing 50% of nitrous oxide (N₂O) and at least 20% of oxygen (O₂) (mol %) according to the invention, preferably an equimolar 50%/50% N₂O/O₂ mixture, is administered by inhalation to said patient.

Depending on the embodiment under consideration, the treatment method of the invention can comprise one or more of the following features:
the gas mixture is administered at least once a day;
the gas mixture is administered at least once a day for 1 to several days, preferably several consecutive days;
the gas mixture is administered over a period of 1 to 3 days;
preferably, the administration is repeated at time intervals of at least 1 week, preferably at least 2 to 4 weeks, depending on the response to the treatment;
the gas mixture is administered for at least 30 min, preferably at least 45 min, advantageously at least 1 hour at each administration, typically between 55 min and 65 min, that is to say for a period sufficient to obtain a long-term effect over several weeks;
the gas mixture is administered at a flow rate of between 1 and 25 l/min, preferably between 2 and 16 l/min;
the patient is an adult or a child (i.e. >2 years old).

According to another aspect, the invention also relates to a pressurized gas container containing a gas mixture according to the invention, in particular an equimolar 50%/50% N₂O/O₂ mixture.

According to yet another aspect, the invention also relates to a system for administering an N₂O/O₂ gas mixture according to the invention, comprising:
a gas container containing the N₂O/O₂ gas mixture to be administered,
a breathing interface for supplying gas to the patient.

Depending on the embodiment under consideration, the administration system according to the invention can comprise one or more of the following features:
the gas container is fitted with a gas distribution valve unit, preferably an integrated regulator valve unit;
the gas container has a cylindrical body;
the gas container has a cylindrical body made of metal or of composite materials, in particular made of aluminum alloy or of steel;
the gas container is fitted with a gas distribution valve unit protected by a protective cover, also called a “cap”, made of polymer or metal;
the breathing interface for supplying gas to the patient is a mask . . . ;
the breathing interface is fluidly connected to the gas container by a flexible hose or the like for conveying the gas;
the gas container contains an N₂O/O₂ gas mixture comprising 50% of nitrous oxide (N₂O) and at least 20% of oxygen (O₂) (mol %), preferably at least 30% of oxygen (O₂), typically an equimolar 50%/50% N₂O/O₂ mixture.

### BRIEF DESCRIPTION OF THE DRAWING

The invention will now be understood more clearly by virtue of the following detailed description, given by way of nonlimiting illustration, in the appended FIG. 1 and the comparative examples set out below, wherein

FIG. 1 shows schematically a system for administering a gas mixture according to the invention.

### DETAILED DESCRIPTION OF PREFERRED EMBODIMENTS

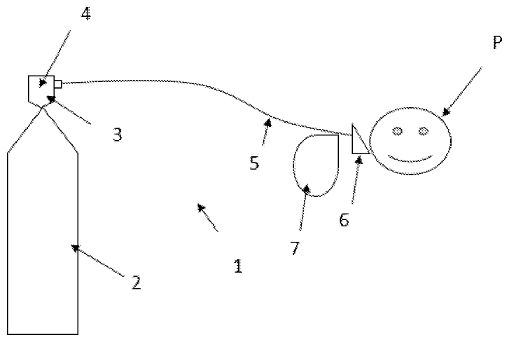

FIG. 1 shows schematically an embodiment of a system for administering 1 an N₂O/O₂ gas mixture according to the invention to a human patient suffering from chronic pain, in particular chronic neuropathic pain, and treated elsewhere with several drug substances, i.e. at least two, from at least two different therapeutic classes which are administered to said patient with the aim of relieving the chronic pain of the latter, but which have little or no efficacy and/or may be poorly tolerated by the patient.

Preferably, the gas mixture consists of 50% of nitrous oxide (N₂O) and 50% of oxygen (O₂), and optionally unavoidable impurities resulting, for example, from the method for producing the gas mixture. This equimolar (50%/50%) N₂O/O₂ binary gas mixture is referred to as EMONO for Equimolar Mixture of Oxygen and Nitrous Oxide.

This administration system 1 comprises a gas cylinder 2 fitted with an integrated regulator valve 3, containing the N₂O/O₂ gas mixture, such as EMONO, which is packaged therein at a pressure that can be up to 300 bar abs, referred to as “high pressure”. The integrated regulator valve 3 makes it possible to reduce the pressure of the N₂O/O₂ gas mixture down to a lower pressure, referred to as “low pressure” or “outlet pressure”, for example of 5 bar abs or less, which is delivered by an outlet connector 4 of the integrated regulator valve 3.

The N₂O/O₂ mixture is conveyed to the patient P, via a flexible pipe 5, which is in fluid communication, on the one hand, with the outlet connector 4 of the integrated regulator valve 3 and, on the other hand, with a gas distribution interface 6, like a breathing mask.

Preferably, a buffer vessel 7, such as a flexible balloon, can be inserted upstream of the breathing interface 6 in order to facilitate the administration of the gas to the patient P.

Of course, it is possible to use other administration devices suitable for supplying this mixture to the patient.

The N₂O/O₂ gas mixture is an inhalable drug used to treat chronic pain, in particular chronic neuropathic pain, of peripheral origin, in patient P who has elsewhere already received several drug substances, i.e. at least two, from at least two different therapeutic classes (i.e. these prior treatments may have been stopped or may be still in progress), which drug substances did not or do not allow the patient's pain to disappear, for example due to a lack of efficacy, in particular a patient refractory to said drug substances.

These drug substances can be, for example, one or more antiepileptics (AEs) of the pregabalin or gabapentin type, one or more antidepressants (ADs) of the fluoxetine type or the like, or one or more opioids, or one or more local anesthetics.

In general, chronic neuropathic pain is distinguished by its pathophysiology and its clinical expression. It has been defined, by the International Association for the Study of Pain, as pain secondary to injury or disease affecting the somatosensory system. It can be of peripheral or central origin.

In the context of the present invention, consideration is preferentially given to chronic neuropathic pain having a peripheral component, also referred to as chronic peripheral neuropathic pain (CPNP). This occurs, for example, as a result of a lesion or irritation, of traumatic, toxic, metabolic, ischemic, immunoallergic or infectious origin, of the peripheral nerves.

It is selected in particular from postherpetic pain, diabetic neuropathy pain, polyneuropathy, and post-traumatic or post-operative pain.

It is characterized by spontaneous and/or provoked pain.

Spontaneous pain may be continuous and/or superficial (e.g. burning sensation) or deep (e.g. feeling of tightening by a vice or compression), and may cause the occurrence of breakthrough pain (i.e. sensation of electric shock) and paresthesia/dysesthesia. Provoked pain is characterized by hypersensitivity to hot, to cold, to touch and is also referred to as “hyperalgia” or “allodynia”.

In order to show the efficacy of the inhalable N₂O/O₂ drug according to the invention, the following comparative examples were carried out.

### Examples

Comparative trials were carried out in 2 countries and in 22 hospitals, using EMONO (50/50% N₂O/O₂) according to the invention and medical air acting as a control (i.e. placebo gas).

These gas mixtures were administered by inhalation to adult patients suffering from chronic peripheral neuropathic pain (CPNP) and already treated (i.e. treatments in progress or having been stopped) with one or more drug substances, in particular at least two drug substances from at least two different therapeutic classes or, by way of comparison, untreated patients, using the same administration system as described in FIG. 1.

Drug substances from at least two different therapeutic classes are one or more opioids, antiepileptics (AEs), antidepressants (ADs) and local anesthetics.

Patients were drawn to receive either EMONO or medical air. However, patients are not told which gas they are inhaling. The patients receive the gases tested while they are awake and conscious (i.e. without any anesthesia).

The treatments were administered for 1 hour per day for 3 consecutive days. Each patient in the study was followed up for 4 weeks and had to complete several questionnaires throughout the follow-up (i.e. NRS, SF-12, NPSI, PGIC) making it possible to assess the change:
in the pain intensity. The NRS questionnaire allows patients to assess the intensity of their pain on a scale ranging from 0 (no pain) to 10 (pain as strong as possible);
in the quality of life. The SF-12 questionnaire represents a quality of life scale. It has 8 dimensions making it possible to assess, over the 4 weeks preceding the administration, the physical component and the mental component of the quality of life of each patient;
in the different components of neuropathic pain. The NPSI questionnaire makes it possible to characterize the different components of the pain. It comprises 12 questions giving a total score and 5 sub-scores representing the following dimensions: spontaneous superficial pain such as burns, spontaneous deep pain such as compression, vice-like sensation, paroxysmal pain such as electric shocks, stabbing, paresthesia and dysesthesia such as tingling, pain caused by friction, and contact with cold;
in the overall painful feeling. The PGIC questionnaire is a scale which makes it possible to measure the change under treatment of the activity limitations, symptoms, emotions and overall quality of life that are related to the painful condition. This change is measured in 7 points ranging from “highly deteriorated” to “highly improved”.

The results of the trials are obtained on patients having received three administrations, either of EMONO or of placebo (i.e. air), including at least one complete administration (i.e. duration of administration ranging from 55 to 65 minutes with cumulative breaks not exceeding 5 minutes), and having done at least 4 assessments of the intensity of their pain during the first week after treatment.

The results are collated in the following tables wherein:

Table 1 collates the results of the change in pain intensity (NRS score) after 1 week of follow-up on the overall population of CPNP patients in the study, and

Table 2 collates the results of the change in pain intensity (NRS score) over the 4 weeks of follow-up in the CPNP patients in the study.

TABLE 1

| | Patients | Patients | |
|---|---|---|---|
| Number of | receiving | receiving a | |
| drug substances | EMONO | placebo (air) | Significance |
| received | (103 patients) | (118) | (P) |
| 0 | 21 patients | 28 patients | p = 0.79 |
| | −0.93 (1.4) | −0.98 (1.5) | Not significant |
| 1 | 36 patients | 33 patients | |
| | −0.76 (1.6) | −0.86 (1.1) | |
| 2 | 33 patients | 42 patients | p = 0.04 |
| | −1.08 (1.2) | −0.54 (1.1) | Significant |
| 3 | 13 patients | 15 patients | |
| | −1.74 (1.8) | −1.13 (1.7) | |

In each cell of Table 1, the following are given: the number of patients involved, the difference in the NRS score between the 1st week following treatment and the week before treatment, and standard deviation of this difference.

It is noted, surprisingly, in view of the results of Table 1, that the patients who were treated with at least two drug substances used to relieve their chronic pain (i.e. opioids, AEs, ADs and/or local anesthetics) have a higher level of response to EMONO than that of the patients who were treated with only one drug substance or than that of those who had no prior treatment for their chronic pain.

In other words, the results obtained show a statistically significant difference in favor of EMONO concerning the reduction in pain intensity for patients treated with EMONO and having received two or three other drug substances used to relieve their chronic pain compared to the other patients, i.e. those treated either with medical air (placebo) regardless of the number of drug substances having been administered to them elsewhere, or EMONO and at most one other drug substance (i.e. 1 or 0).

This is surprising since, in general, the level of response of naive or barely treated patients is usually better than that of extensively treated patients. However, in this case it is the opposite.

This demonstrates the efficacy of the administration by inhalation of a gas mixture according to the invention containing 50% of nitrous oxide (N₂O) and at least 20% of oxygen (O₂) (mol %) to patients treated elsewhere with several drug substances, i.e. at least two drug substances, which belong to different therapeutic classes, namely AEs, ADs, opioids or local anesthetics, in order to treat their chronic pain, in particular peripheral neuropathic pain.

TABLE 2

| | Patients receiving | Patients receiving |
|---|---|---|
| Number of | at least 2 drug | 0 or 1 drug |
| weeks after | substances and who | substance and who |
| administration | are 30% responders | are 30% responders |
| of EMONO | (*) (46 patients) | (*) (57 patients) |
| 1 | 13/46 (28.3%) | 15/57 (26.3%) |
| 2 | 15/46 (32.6%) | 11/55 (20.0%) (**) |
| 3 | 16/46 (35.6%) | 14/55 (25.5%) (**) |
| 4 | 13/46 (28.9%) | 12/54 (22.2%) (**) |

(*) Number of patients who are 30% responders, that is to say for whom there is a 30% drop in their NRS score.
(**) the questionnaires of 2 or 3 patients could not be assessed or could not be used.

Table 2 makes it possible to compare the efficacy of EMONO in patients treated with 0 to 3 other drug substances such as AEs, ADs, opioids or local anesthetics, more particularly in patients with a 30% drop in their NRS score.

The results obtained clearly show a higher proportion (%) of patients attesting to a reduction in the intensity of their chronic pain from week 1 to week 4, in the group of patients according to the invention, that is to say who have received elsewhere at least 2 drug substances belonging to different therapeutic classes (i.e. AEs, ADs, opioids, local anesthetics).

This difference appears from the start, becomes more significant from the second week and continues for 4 weeks.

These results are surprising for several reasons.

First of all, they confirm that the drug according to the invention containing 50% of N₂O has a targeted efficacy in combating pain in a particular (sub)population of patients, namely patients treated with several drug substances (i.e. AEs, ADs, opioids, local anesthetics), while its efficacy is less in those treated with a single drug substance or none.

They then demonstrate a persistence of the efficacy of the inhalable drug according to the invention, in this case EMONO, for a period of several weeks, namely in this case 4 weeks, in these same patients after a short administration. This second surprising effect does not exist with any of the other systemic background treatments received by the patients.

Finally, it should be emphasized that none of these patients showed a secondary reaction to EMONO, that is to say that the tolerance of EMONO is satisfactory in this (sub)population of patients, i.e. patients treated by several drug substances from different classes (i.e. AEs, ADs, opioids, local anesthetics).

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A method of treating chronic pain in a human patient previously treated for chronic pain with drug substances from at least two different therapeutic classes, the method comprising administering a gas mixture containing 50% of nitrous oxide (N₂O) and at least 20% of oxygen (O₂) (mol %) by inhalation by the human patient.

2. The method of claim 1, characterized in that the chronic pain of the human patient is refractory to drug substances from at least two different therapeutic classes.

3. The method of claim 1, characterized in that the gas mixture contains at least 30% of oxygen, preferably at least 40% of oxygen (mol %).

4. The method of claim 3, characterized in that the gas mixture contains oxygen, N₂O and nitrogen (N₂).

5. The method of claim 3, characterized in that the gas mixture consists of 50% of nitrous oxide (N₂O) and 50% of oxygen (O₂) (mol %).

6. The method of claim 2, characterized in that the drug substances from at least two different therapeutic classes are selected from opioids, antiepileptics (AEs), antidepressants (ADs) and local anesthetics.

7. The method of claim 6, characterized in that the drug substances from at least two different therapeutic classes comprise:
an antiepileptic compound selected from gabapentin and pregabalin,
an antidepressant compound:
of the tricyclic type, preferably nortriptyline, desipramine or amitriptyline, or
of the serotonin-norepinephrine reuptake inhibitor type, preferably duloxetine,
an opioid compound selected from tramadol, codeine, oxycodone, tapentadole, fentanyl, remifentanyl and morphine, and/or
a local anesthetic compound selected from lidocaine and capsaicin.

8. The method of claim 1, characterized in that the chronic pain is neuropathic pain.

9. The method of claim 8, characterized in that the chronic pain is peripheral neuropathic pain.

10. The method of claim 9, characterized in that the chronic peripheral neuropathic pain results from, or is associated with, a lesion or irritation of one or more peripheral nerves.

11. The method of claim 1, characterized in that the patient is an adult, a teenager or a child.

12. The method of claim 1, characterized in that the gas mixture is administered by inhalation at least once a month for a period of administration of at least 30 min.

13. The method of claim 12, characterized in that the gas mixture is delivered for inhalation at a flow rate of between 1 and 25 l/min.

14.-15. (canceled)
